(19) United States
(12) Patent Application Publication
(10) Pub. No.: US 2006/0093562 A1
(43) Pub. Date: May 4, 2006

(54) Use of sodium decyl sulfate in toothpaste

(76) Inventors: Kenneth E. Kellar, Flemington, NJ (US); Justyna Nicinska, Princeton, NJ (US)

Correspondence Address: Church & Dwight Co., Inc., Law Dept. - Patents, 469 North Harrison Street, Princeton, NJ, 08543-5297, US

(21) Appl. No.: 10/981,242

(22) Filed: Nov. 4, 2004

(51) Int. Cl. A61K 8/21

(52) U.S. Cl. 424/49; 424/52

(57) ABSTRACT

Sodium decyl sulfate yields high foam production when added to aqueous compositions containing high levels of sodium bicarbonate. Sodium bicarbonate toothpastes and gels containing sodium decyl sulfate have high foaming properties and hold a desirable ribbon-type shape when applied to a toothbrush. Rheological properties and phase stability of such toothpastes can be controlled by the addition of potassium carbonate.

### FIELD OF THE INVENTION

The present invention is directed to toothpastes which contain baking soda and to providing stable toothpaste formulations containing same.

### BACKGROUND OF THE INVENTION

The use of bicarbonate salts (baking soda) as a dentifrice or the incorporation of such salts into dentifrice compositions is well known in the art of oral care. A renewed interest in incorporating bicarbonate salts into toothpaste has emerged in light of the success of the present assignee's Dental Care® and PeroxiCare® products. The addition of bicarbonate salts into dentifrices is beneficial for several reasons such as for providing good plaque removing capabilities, as well as for improving the whitening properties of dentifrices. Importantly, bicarbonate salts provide a clean fresh feeling in the oral cavity after brushing and rinsing with water.

One type of dentifrice that has recently been described in the patent literature and introduced into the market is a toothpaste product which functions to remineralize teeth. Certain of these products also contain bicarbonate salts. For a full description of demineralization of teeth and remineralizing products, see commonly assigned co-pending U.S. patent application Ser. No. 10/686,879 entitled “Prevention of Crystal Formation in Toothpaste” filed Oct. 16, 2003 (specifically, pages 2-3); commonly assigned co-pending U.S. patent application Ser. No. 10/686,911 entitled “Tooth Enamel Rejuvenating Toothpaste” filed Oct. 16, 2003 (specifically, pages 1-3); and commonly assigned co-pending U.S. patent application Ser. No. 10/882,063 entitled “Stable Baking Soda/Peroxide with Calcium and Phosphate Whitening Product” filed Jun. 30, 2004 (specifically, pages 4-7), all of which are incorporated herein by reference.

Oral compositions such as dentifrices, because of their intended use in the mouth, should preferably taste good. Dentifrices with high foaming properties are beneficial for improved cleaning and ensuring a pleasant mouth feel. Furthermore, for dentifrices, particularly toothpastes, it is desired that a creamy or gel mass be smoothly extrudable from a collapsible tube. Smooth extrusion from a tube is one of the important requirements for dentifrices. For remineralization toothpastes, in particular toothpastes packaged in a divided tube, it is important that each portion of the divided composition be extruded evenly from the tube to provide maximum benefit. With such remineralizing dentifrice compositions containing reactive components of calcium, phosphate, and fluoride salts, and further containing additional salts such as bicarbonates to enhance oral activity and provide a clean mouth feel, aging of the composition often deteriorates the even extrudability of the divided composition. On the other hand, it is most useful if the toothpaste has a consistency such as to hold a ribbon-type shape for a significant length of time when applied to a brush, and, it is important for the toothpaste not to undergo phase separation at elevated temperatures (temperatures above room temperature). For toothpaste formulations considered herein, these desired qualities of foam production, body and stand-up, and phase stability are very difficult if not impossible to obtain with surfactants currently in use in oral care products. For example, sodium lauryl sulfate (SLS) cannot be used with bicarbonate and calcium salts at the salt levels pertinent to formulations described herein, for such salts render the SLS insoluble and bicarbonate salt alone is enough to cause SLS to produce very little foam. Sodium lauroyl sarcosinate (SLOS), although it produces significant foam, causes bicarbonate-containing dentifrices to undergo phase separation, even at room temperature. Moreover, combinations of SLS and SLOS cannot produce a paste phase that produces significant foam and remains phase stable at room temperature. Another commercial surfactant approved for use in oral care, cocamidopropyl betaine (CBT), will produce significant foam in the presence of bicarbonate, but loses that ability when exposed to calcium ions. Furthermore, CBT causes bicarbonate-containing formulations to undergo phase separation, even at room temperature. Additionally, combinations of SLS and CBT cannot produce a paste phase that produces significant foam and remains phase stable at room temperature. One surfactant, sodium decyl sulfate (SDS), has been found that gives high foam production without causing phase separation at room temperature.

### SUMMARY OF THE INVENTION

It has now been found that the presence of sodium decyl sulfate (SDS) as a toothpaste ingredient provides for increased foam production and allows the toothpaste to hold a desirable ribbon-type shape for a significant length of time when applied to a toothbrush. Sodium decyl sulfate has particular use in sodium bicarbonate-containing dentifrice compositions and, in particular, in bicarbonate-containing remineralizing compositions which are formed from divided calcium/phosphate formulations which are dispensed simultaneously from a container or tube. Potassium carbonate may also be used to prevent a bicarbonate-containing dentifrice containing SDS from undergoing phase separation at elevated temperatures (100° F. or higher). The potassium carbonate also prevents the SDS from making the paste phase too thick. That SDS increases the body and stand-up of the toothpaste is no longer an advantage when it becomes difficult to extrude the dentifrice. However, the presence of potassium carbonate makes it easier to extrude the paste phase.

### DETAILED DESCRIPTION OF THE INVENTION

In accordance with this invention, sodium decyl sulfate (SDS) is incorporated into sodium bicarbonate-containing dentifrices to provide improved foaming and thickening properties. While having particular use in dentifrices, SDS can also be used as a surfactant in high salt (sodium bicarbonate) scrubbing products such as, for example, pot and dish scrubbers, hard surface cleaners such as for kitchen or bathroom surfaces, etc. SDS is preferred for use in cleaners in which high dilution with water is unnecessary or unwanted. SDS has preferred use in compositions which are diluted with water in an amount that is less than eight times the solids content of the composition. Compositions that need to be diluted with water in an amount that is no more than four times the solids content of the composition are particularly preferred.

The dentifrices of the present invention include toothpastes, dental creams, or dental gels. These dentifrices comprise the several essential, as well as optional, components disclosed hereinafter. A dentifrice is a substance or preparation used with a toothbrush to aid mechanical cleaning of the accessible surfaces of the teeth. A typical formulation for a dentifrice (e.g., toothpaste) contains varying amounts of humectants, organic thickeners and gums, inorganic thickeners, and flavors and sweeteners. Most dentifrices contain one or more active components to reduce decay, reduce or remove tartar buildup, reduce sensitivity, or provide for remineralization.

With respect to dentifrices in which SDS can be used effectively, of particular interest are those containing sodium bicarbonate. Sodium bicarbonate can be incorporated into dentifrices in an amount of about 20 to 65%, preferably within the range of about 30 to 60%, by weight, and it is in connection with these bicarbonate containing dentifrices that the instant invention has particular importance.

Organic surface-active agents are used in the dentifrices of the present invention to achieve increased cleaning action and improve the detergent and foaming properties of the dentifrices. Organic surfactants which may be so utilized can be anionic, nonionic or ampholytic in nature.

SDS is an anionic surfactant used in the dentifrices of this invention. Other suitable surface-active materials which can be added in addition to SDS include nonionic agents such as condensates of sorbitan monostearate with ethylene oxide, condensates of ethylene oxide with propylene oxide or, condensates of propylene glycol (available under the trademark “Pluronics”). Other examples of water-soluble nonionic surfactants useful in the dentifrices of the present invention are the condensation products of ethylene oxide with various other compounds which are reactive therewith and have long hydrophobic chains (e.g. aliphatic chains of about 12 to 20 carbon atoms), which condensation products (“ethoxamers”) contain hydrophilic polyoxyethylene moieties, such as condensation products of poly(ethylene oxide) with fatty acids, fatty alcohols, fatty amides, or polyhydric alcohols (e.g., sorbitan monostearate).

In the instant invention, the sodium decyl sulfate surfactant is preferably utilized alone, or in admixture with one or more nonionic or ampholytic surfactants. In toothpastes made according to this invention, the amount of the SDS surfactant used is preferably within the range of about 0.05% to about 5%, more preferably from about 0.5% to about 2.0% by weight. Additional nonionic or ampholytic surfactants can be present in amounts of about 0.05% to about 3.0% by weight.

In a toothpaste made according to this invention, the liquid vehicle comprises water and humectant, typically in an amount ranging from about 10% to about 90% by weight of the preparation. Glycerin, propylene glycol, sorbitol, polypropylene glycol and/or polyethylene glycol (e.g., molecular weight of 400-600) exemplify suitable humectants/carriers. Also advantageous are liquid mixtures of water, glycerine and sorbitol. In translucent gels, where the refractive index is an important consideration, it is preferred to use higher ratios of humectant to water than in opaque pastes.

Toothpastes, creams and gels made according to this invention typically also contain a natural or synthetic thickener or gelling agent in proportions of about 0.1% to about 10%, preferably about 0.5% to about 5%, by weight. Suitable organic thickeners include sodium carboxymethyl cellulose, gum tragacanth, starch, carrageenan, polyvinylpyrrolidone, hydroxyethylpropyl cellulose, hydroxybutyl methyl cellulose, hydroxypropyl methyl cellulose, or hydroxyethyl cellulose, and are usually used in concentrations of 0.1-2.0%. Inorganic thickeners such as hydrated silicas may also be used at levels of about 0.5-10%.

Suitable flavoring and sweetening agents may also be employed in the dentifrices of the invention. Examples of suitable flavorants include the flavoring oils, for example, oils of spearmint, peppermint, wintergreen, sassafras, clove, sage, eucalyptus, marjoram, cinnamon, lemon and orange, as well as methylsalicylate. Suitable sweeteners include sodium cyclamate, perillartine, saccharin, sodium saccharin and ammoniated glycyrrhizin (e.g., its monoammonium salt), and the like. Suitably, the flavoring and sweetening agent together comprise from about 0.01% to 5% or more by weight of the dentifrice. Preferably, the amount of flavoring oil is above 0.3%, e.g. 0.8 to 1.2%.

Various other materials may be incorporated in the dentifrices of this invention. Examples thereof are coloring and whitening agents, preservatives, silicones, chlorophyll compounds, and mixtures thereof, and other constituents. These adjuvants are incorporated in the instant compositions in amounts which do not substantially adversely affect the properties and characteristics desired and are selected and used in effective amounts, depending upon the particular adjuvant and type of preparation involved.

The dentifrice of this invention can be of the mineralizing/remineralizing type initially described by Winston and Usen, as noted previously. The aspects of these remineralizing dentifrices which result in the particular need for the present invention include:
1. The high bicarbonate content, which prevents foam production with many surfactants conventionally used in oral care products.
2. The high calcium content, which also prevents foam production with most surfactants conventionally used in oral care products.
3. The dual-chamber tube, which makes it necessary to include potassium carbonate to help extrude the paste phase through one of the chambers when SDS is included as the foam-inducing surfactant.

Further, according to the present invention, it has been found that using potassium carbonate assists in maintaining the stability of the dentifrice, i.e., no phase separation, at room temperature and at 100° F. when SDS is used as the surfactant. Levels of the carbonate salts can range from up to about 5 wt. % of the phosphate-containing part, typically up to about 2 wt. %. It has also been found that the inclusion of potassium carbonate can decrease the body and stand-up of the formulation to a degree that makes the paste phase easy to extrude.

### EXAMPLES

### Example 1

Tables 1 and 2 illustrate a useful remineralization toothpaste of this invention. Table 1 is directed to the phosphate-containing part, while Table 2 sets forth the cationic calcium part.

TABLE 1

| Raw Material | Percent (by weight) |
|---|---|
| Glycerine | 26.810 |
| Sodium Carboxymethyl Cellulose | 0.500 |
| Purified Water | 9.306 |
| Sodium Fluoride | 0.440 |
| Sodium Saccharin | 1.000 |
| Potassium Carbonate (Anhydrous) | 1.800 |
| Sodium Bicarbonate, Grade 3 | 54.000 |
| Potassium Phosphate Dibasic Anhydrous | 1.800 |
| Sodium Decyl Sulfate (37.38% Solution) | 3.344 |
| Flavor | 1.000 |
| Total | 100 |

TABLE 2

| Raw Material | Percent (by weight) |
|---|---|
| Glycerin | 34.550 |
| Sodium Carboxymethylcellulose | 0.500 |
| Xanthan Gum | 0.300 |
| Methylparaben | 0.050 |
| Propylparaben | 0.050 |
| Sorbitol HSC (70% Sol.) | 29.297 |
| Purified Water | 8.750 |
| Calcium Sulfate | 4.000 |
| Sodium Sulfate | 3.000 |
| Sodium Saccharin | 0.500 |
| FD&C Blue #1 | 0.003 |
| Silicon Dioxide (Aerosil 200VS) | 2.000 |
| Sylodent 700 (Hydrated Silica) | 14.000 |
| Sodium Decyl Sulfate (37.38% solution) | 2.000 |
| Flavor | 1.000 |
| Total | 100 |

### Example 2

The foam height achieved using various surfactants in the toothpaste composition of Table 1 is shown in Table 3. The test used 1% total surfactant (actives). The surfactant was added to an aqueous mixture containing 1 g of surfactant-free paste of Table 1, 1 g of the surfactant-free version of the gel of Table 2, and 6 g of water. The mixture, prepared in a centrifuge tube, is placed on a Vortex-Genie 2 (Scientific Industries) on setting 5, and allowed to vortex for 1 minute. The total height measured was the total volume of the foam plus any liquid underneath. The liquid height was the volume of liquid underneath the foam. Liquid volumes of less than 5 mL could not be quantified since the lowest level on the centrifuge tube used to conduct the foam test was 5 mL.

TABLE 3

| | Abbreviation | Total | Liquid |
|---|---|---|---|
| | for | Height | Height |
| Surfactant | Surfactant | (mL) | (mL) |
| *Sodium Lauryl Sulfate | SLS | 9 | 7 |
| (Sodium Dodecyl Sulfate) | | | |
| *Sodium Dodecylbenzene Sulfonate | SDDBS | 8 | 7 |
| Sodium Decylbenzene Sulfonate | SDBS | 8 | 7 |
| Sodium Octylbenzene Sulfonate | SOBS | 8 | 7 |
| Sodium Decyl Sulfate | SDS | 19 | <5 |
| Sodium Laureth Sulfate, 1 Ether Group | SLES-1 | 9 | 7 |
| Sodium Laureth Sulfate, 2 Ether Groups | SLES-2 | 9 | 7 |
| Sodium Laureth Sulfate, 3 Ether Groups | SLES-3 | 10 | 7 |
| *Sodium Lauroyl Sarcosinate | SLOS | 14 | <5 |
| *Cocamidopropyl Betaine | CBT | 10 | 7 |
| ***Alkyl Polyglycosides | APG-625 | 8 | 7 |
| Glucopon 625 UP ® | | | |
| ***Alkyl Polyglycosides | APG-600 | 0 | 8 |
| Glucopon 600 UP ® | | | |
| Alkylpolyglucoside Carboxylate | APC | 9 | 7 |
| **Fatty Acids, Coco, 2-Sulfoethyl | FAC2S | 9 | 7 |
| Esters, Sodium Salts | | | |
| ELFAN ® AT 84 G | | | |

Used in oral products in United States (*)
Used in oral products in Europe (**)
Reported to have GRAS status by manufacturer (***)

Based on the results in Table 3, the highest foam production occurred when SDS was used.

### Example 3

The presence of SDS increases foaming and also increases body and stand-up toothpastes. Four formulations of a paste phase similar to that of Table 1 were investigated. The first was a paste prepared containing all SLS (1.25%). The second was a laboratory preparation containing 0.85% SLS and 0.4% SLOS. The third was a laboratory preparation containing all SDS (1.25%). The fourth was a laboratory preparation containing all SDS (2%). Visually, when placed on a toothbrush, the formulation containing the SLOS was the most runny, and this was followed by the paste containing all SLS. The formulation containing 2% SDS was less was runny than the formulation containing 1.25% SDS. The least runny formulations contained all SDS, and these formulations also had much better stand-up and body than either of the other two formulations. Confirmation of these visual results was accomplished by measuring the shear rate versus shear stress of the different paste phases.

### Example 4

A foam test was run comparing the foam generated by the following two preparations:
1. Table 2 gel, no surfactant. Table 1 paste, 2% SDS.
2. Table 2 gel, 0.75% SDS. Table 1 paste, 1.25% SDS.

Both preparations were found to give the same foam height, 18-19 mL total height with less than 5 mL liquid. Furthermore, the gel used to conduct this test was prepared about four months before running the foam test, giving adequate time for the binding of SDS and calcium to take place. The SDS used, Polystep B-25 (Stepan), contained only about 75% of C10 (decyl), and the remaining 25% is C12 (lauryl, or dodecyl). Consequently, it appears that all of the SDS does not have to be placed in the paste phase.

### Example 5

Solutions containing about 26.8% glycerine and 11.4% water were prepared and saturated with sodium bicarbonate. A saturated solution occurred at about 3.8% sodium bicarbonate. The solutions were filtered to remove any particulate sodium bicarbonate. Surfactant (SLS, SLOS, or SDS) was added to the saturated sodium bicarbonate solutions to give an active content corresponding to 1.25% in the paste phase. The solutions were stored at room temperature and at 100° F., and the results are given in Table 4. Also given in Table 4 are the stability results for paste formulations of Table 1 prepared with the corresponding surfactant. These results clearly show that phase separation in the paste phase only occurs for surfactants that are soluble in the saturated sodium bicarbonate solution. Of particular relevance is that the paste prepared with SDS is stable at room temperature but not at 100° F., and that SDS is insoluble in the saturated sodium bicarbonate solution at room temperature, but soluble at 100° F.

TABLE 4

| | SLS | SLOS | SDS |
|---|---|---|---|
| | Phase Behavior | Phase Behavior | Phase Behavior |
| | Solubility | Solubility | Solubility |
| RT | Single Phase | Separation | Single Phase |
| | Turbid | Clear | Turbid |
| 100° F. | Single Phase | Separation | Separation |
| | Turbid | Clear | Clear |

### Example 6

It was believed the use of potassium bicarbonate or potassium carbonate could provide the necessary insolubility of SDS. These potassium salts are more soluble than the corresponding sodium salts, so that the SDS would be more effectively salted out of solution. Furthermore, the potassium salt of decyl sulfate (KDS) is less soluble than SDS. KDS would be expected to form to some extent in the presence of potassium carbonate and potassium bicarbonate.

In saturated potassium bicarbonate solutions of glycerol/water (as above), SDS is insoluble at room temperature, 100° F., and 122° F. However, potassium salts can have an undesirable effect on flavor due to an interaction with saccharin. Therefore, it is preferred to use as little potassium bicarbonate or potassium carbonate as possible to salt out the SDS. Since carbonates are more effective at salting out surfactants, potassium carbonate was substituted for sodium carbonate, and found that solutions containing 134.06 g glycerin, 46.54 g distilled water, and 9.00 g potassium carbonate (anhydrous) were clear and homogeneous. However, when SDS was added corresponding to 1.25% in the paste phase (3.34 g of 37.38% SDS solution), the solutions were turbid at room temperature, 100° F., and 122° F. If this same procedure is repeated using sodium carbonate, SDS is insoluble at room temperature but soluble at 100° F. Consequently, since SDS is insoluble when potassium carbonate is substituted for sodium carbonate, a paste phase prepared with potassium carbonate (no sodium carbonate) would be phase stable at room temperature, 100° F., and 122° F.

A paste phase formulation was prepared according to the percentages given in Table 1. Samples of this formulation were taken, and placed at room temperature and at 100° F. After at least three months, no phase separation occurred for either storage condition. The viscosity of this formulation appeared visually to be similar to that of the original paste phase that did not contain potassium carbonate. Thus, the substitution of potassium carbonate for sodium carbonate in the paste phase allowed for stability at higher temperatures (100° F.) and also resulted in a paste phase that has a viscosity similar to that of the original formulation. Foam production was not affected by this substitution.

What is claimed is:

1. An aqueous, cleaning composition containing at least 20 wt. % alkali metal bicarbonate and a surfactant comprising sodium decyl sulfate.

2. The cleaning composition of claim 1 wherein said alkali metal bicarbonate is sodium bicarbonate.

3. The cleaning composition of claim 2 in the form of a dentifrice.

4. The composition of claim 3 containing a humectant.

5. The composition of claim 3 further containing a source of fluoride ions.

6. The composition of claim 5 wherein said source of fluoride ions is sodium fluoride.

7. The composition of claim 5 containing 0.05 to about 5% by weight of said sodium decyl sulfate.

8. The composition of claim 7 containing from about 0.5 to 2.0% by weight sodium decyl sulfate.

9. The composition of claim 4 wherein said humectant is glycerin.

10. The composition of claim 3 comprising a first discrete calcium-containing part containing at least one partially water soluble calcium salt, and a second discrete phosphate-containing part containing a water soluble orthophosphate salt, a water soluble fluoride salt, and said sodium bicarbonate.

11. The composition of claim 10 wherein said sodium decyl sulfate is contained in both of said first and second parts.

12. The composition of claim 10 wherein the total amount of sodium decyl sulfate in said composition is within the range of from about 0.05 to about 5% by weight.

13. The composition of claim 10 wherein the total amount of sodium decyl sulfate in said composition is within the range of from about 0.5 to about 2% by weight.

14. The composition of claim 10 wherein both said first and second parts contain a humectant.

15. The composition of claim 10 wherein said sodium decyl sulfate is present in said second discrete phosphate-containing part.

16. The composition of claim 15 wherein said second discrete phosphate-containing part contains an alkali metal carbonate.

17. The composition of claim 16 wherein said alkali metal carbonate is potassium carbonate.

18. A method of cleaning a surface with an aqueous cleaning composition containing at least 20% by weight sodium bicarbonate and a surfactant comprising sodium decyl sulfate.

19. The method of claim 18 wherein said cleaning composition is diluted with water in an amount of no more than eight times the solid content of said composition.

20. The method of claim 18 wherein said cleaning composition is diluted with water in an amount of no more than four times the solid content of said composition.
